(19) United States
(12) Patent Application Publication
(10) Pub. No.: US 2022/0258185 A1
(43) Pub. Date: Aug. 18, 2022

(54) MICROFLUIDIC DEVICE AND METHOD FOR MANUFACTURING THE SAME

(71) Applicant: SUZHOU SKYWELL HEALTHCARE INFORMATION CO., LTD., Suzhou, Jiangsu (CN)

(72) Inventor: Yu GU, Suzhou, Jiangsu (CN)

(21) Appl. No.: 17/619,244

(22) PCT Filed: Jun. 15, 2020
(86) PCT No.: PCT/CN2020/096094

§ 371 Date: Dec. 14, 2021

(30) Foreign Application Priority Data
Jun. 17, 2019 (CN) 201910521085.0

(51) Int. Cl. B05B 1/26; B81B 1/00; B81C 1/00

(52) U.S. Cl. CPC B05B 1/26; B81B 1/002; B81C 2201/0143; B81B 2201/057; B81B 2203/0338; B81C 1/00071

(57) ABSTRACT

A microfluidic device comprising: a first substrate (402,502,602,702,802) having a first assembling side (402a,702a, 802a); and a second substrate (404,504,604,704,804) having a second assembling side (404a, 504a, 604a, 804a) connectable with the first assembling side (402a,702a, 802a) to assemble the first substrate (402,502,602,702,802) and the second substrate (404,504,604,704,804) together. At least one of the first assembling side (402a,702a, 802a) and the second assembling side (404a, 504a, 604a, 804a) has a fluid chamber channel (406,706,806), and after the first substrate (402,502,602,702,802) and the second substrate (404,504,604,704,804) are connected together, the fluid chamber channel (406,706,806) forms a fluid chamber having a fluid inlet (408,608,708,808) and a fluid outlet (410,510,610,710,810). The at least one of the first assembling side (402a,702a, 802a) and the second assembling side (404a, 504a, 604a, 804a) having the fluid chamber channel (406,706,806) has an outlet expansion groove (418,518,618,718,818, 818) adjacent to and extending downstream from the fluid outlet (410,510,610,710,810), and wherein at the fluid outlet (410,510,610,710,810), an outer peripheral profile of the outlet expansion groove (418,518,618,718,818, 818) is located outside an outer peripheral profile of the fluid outlet (410,510,610,710,810).

### TECHNICAL FIELD

The present application relates to the technical field of microfluidics, and more particularly, to a microfluidic device and its manufacturing method.

### BACKGROUND

Microfluidic technology is a technology for precisely controlling and manipulating small volumes of fluids. In practical applications, the dimensions of fluid channels in microfluidic devices that implement microfluidics are very small, ranging from about 500 micrometers to 100 nanometers, or even smaller.

With the continuous development of related research, microfluidic technology has been applied in many fields. The inkjet print head is one of the most successful commercial applications of microfluidic technology. In addition, some liquid atomizers, especially medical atomizers with strict requirement on volume control, have gradually adopted microfluidic devices as their atomizing nozzles. Subject to high pressure, the atomizing nozzle can atomize liquid into very tiny droplets to increase the absorption rate of the droplets in lungs.

However, existing microfluidic devices have limited precision control over the fluid volume or flow rate, and thus an improved microfluidic device is needed.

### SUMMARY

An objective of the present application is to provide a microfluidic device to improve precision of fluid volume and flow rate dispensed through the microfluidic device.

In one aspect of the present application, a microfluidic device is provided. The microfluidic device comprises: a first substrate having a first assembling side; and a second substrate having a second assembling side connectable with the first assembling side to assemble the first substrate and the second substrate together. At least one of the first assembling side and the second assembling side has a fluid chamber channel, and after the first substrate and the second substrate are connected together, the fluid chamber channel forms a fluid chamber having a fluid inlet and a fluid outlet. The at least one of the first assembling side and the second assembling side having the fluid chamber channel has an outlet expansion groove adjacent to and extending downstream from the fluid outlet, and wherein at the fluid outlet, an outer peripheral profile of the outlet expansion groove is located outside an outer peripheral profile of the fluid outlet.

In another aspect of the present application, a method for manufacturing a microfluidic device is provided. The method comprises: providing a first substrate having a first assembling side; providing a second substrate having a second assembling side; forming, on the first assembling side, a plurality of fluid chamber channels each having a fluid inlet and a fluid outlet; forming, on the first assembling side, a fluid expansion groove adjacent to and extending downstream from each fluid outlet, and wherein at each fluid outlet, an outer peripheral profile of the outlet expansion groove is located outside an outer peripheral profile of the fluid outlet; connecting the first assembling side of the first substrate with the second assembling side of the second substrate to assemble them together, such that the plurality of fluid chamber channels form a plurality of fluid chambers, respectively; and scribing the first substrate and the second substrate at each outlet expansion groove to separate the plurality of the fluid chambers.

The above is an overview of the application, and there may be simplifications, generalizations, and omissions of details, so those skilled in the art should realize that this section is only illustrative and is not intended to limit the scope of the application in any way. This summary section is neither intended to determine the key features or essential features of the claimed subject matter nor intended to be used as an auxiliary means to determine the scope of the claimed subject matter.

### BRIEF DESCRIPTION OF THE DRAWINGS

The above and other features of the content of the present application will be more fully understood through the following description and the appended claims in combination with the drawings. It can be understood that these drawings only depict several embodiments of the content of the present application, and therefore should not be considered as limiting the scope of the content of the present application. By adopting the drawings, the content of the present application will be explained more clearly and in detail.

FIG. 1 illustrates a partial schematic view of a microfluidic device used as an atomizing nozzle at its liquid outlet;

FIG. 2a illustrates a wafer surface near a scribing groove after being cut by a diamond slicer;

FIG. 2b illustrates a wafer surface near a scribing groove after being cut by a laser;

FIGS. 3a to 3c illustrate several schematic diagrams of uneven edges of fluid outlets caused by scribing defects;

FIG. 3d illustrates a simulated profile of a spray emitted from a microfluidic device with the scribing defect shown in FIG. 3a;

FIGS. 4a to 4c illustrate schematic diagrams of a microfluidic device 400 according to an embodiment of the present application;

FIG. 5a illustrates perspective views of a first substrate and a second substrate when a wafer including a plurality of microfluidic devices as shown in FIG. 4a is not sliced;

FIG. 5b illustrates an assembling side of the second substrate shown in FIG. 5a;

FIG. 5c illustrates that the first substrate and the second substrate shown in FIGS. 5a and 5b overlap with each other;

FIGS. 6a to 6c illustrate schematic diagrams of a microfluidic device 600 according to another embodiment of the present application;

FIG. 7 illustrates a schematic diagram of a microfluidic device 700 according to another embodiment of the present application;

FIG. 8 illustrates a schematic diagram of a microfluidic device 800 according to another embodiment of the present application;

FIG. 9 illustrates a method 900 for manufacturing a microfluidic device according to yet another embodiment of the present application.

### DETAILED DESCRIPTION

In the following detailed description, reference is made to the drawings that form a part thereof. In the drawings, similar symbols generally indicate similar components, unless context dictates otherwise. The illustrative embodiments described in the detailed description, drawings, and claims are not meant to be limiting. Other embodiments may be adopted and other changes may be made without departing from the spirit or scope of the subject matter of this application. It can be understood that various aspects of the content of this application, which are generally described in this application and illustrated in the drawings, can be configured, replaced, combined, and designed in various configurations, all of which clearly constitute the content of this application.

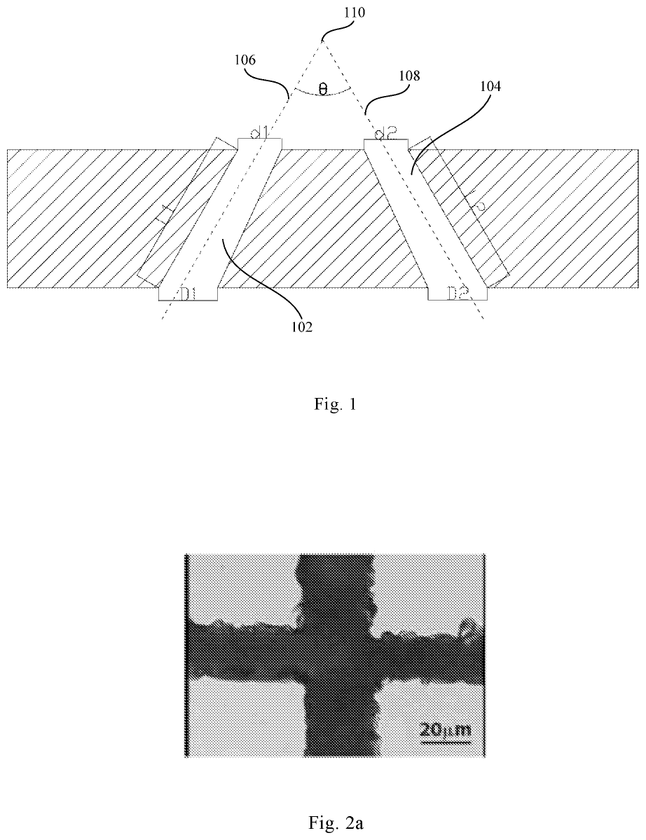

FIG. 1 illustrates a partial schematic view of a microfluidic device used as an atomizing nozzle at its liquid outlet.

As shown in FIG. 1, the microfluidic device has two fluid channels 102 and 104 at the liquid outlet, and these two liquid channels 102 and 104 form jet flows 106 and 108, respectively. The two jet flows 106 and 108 can meet at a convergence point 110 outside the microfluidic device, thereby atomized into tiny droplets due to mutual collision. Ideally, the fluid channel 102 has an inlet diameter D1, an outlet diameter d1, and a channel length L1, while the fluid channel 104 has an inlet diameter D2, an outlet diameter d2, and a channel length L2. These structural parameters may significantly affect the atomization pressure, atomization flow rate, atomization cone angle and atomization particle size of the spray formed by and emitted from the microfluidic device, thus it is required to adopt manufacturing processes with extremely high precision to manufacture this microfluidic device.

In actual mass production, the microfluidic device shown in FIG. 1 can generally be mass-produced using a microfabrication process. For example, a plurality of repeated cell structures of microfluidic devices arranged in an array can be formed on a silicon wafer, a glass substrate or a wafer of other materials through a microfabrication process, and then the wafer can be cut through a scribing process to separate the respective unit structures of microfluidic devices. The inventor of the present application found that for microfluidic devices manufactured by microfabrication processes, although the internal structural parameters of such devices can be accurately controlled by processes such as photolithography and etching, the manufactured devices actually still have significant variations in performance. Many of the devices in the same batch of production do not meet design standards and requirements. This leads to a low yield of mass-produced microfluidic devices.

After further research, the inventor found that the above variations in the actual performance of the microfluidic devices are mainly due to the low precision of the scribing process. Specifically, the wafer scribing generally adopts a mechanical diamond scribing process, which uses a high-hardness diamond slicer to cut at the scribing lines of a wafer at a high speed to form slice marks. At the same time, a worktable carrying the wafer moves linearly at a certain speed along a tangential direction of a contact point between the diamond slicer and the wafer, so that the wafer can split at the slice marks into individual microfluidic devices. However, cutting hard and brittle silicon or glass wafers by diamond slicers is prone to generate mechanical stress. The narrower the scribing lines are, the greater the stress at regions adjacent to the scribing lines is, causing defects such as chipping, micro-cracks, delamination, etc. at the edges of the devices. And such defects may directly affect the performance of the devices.

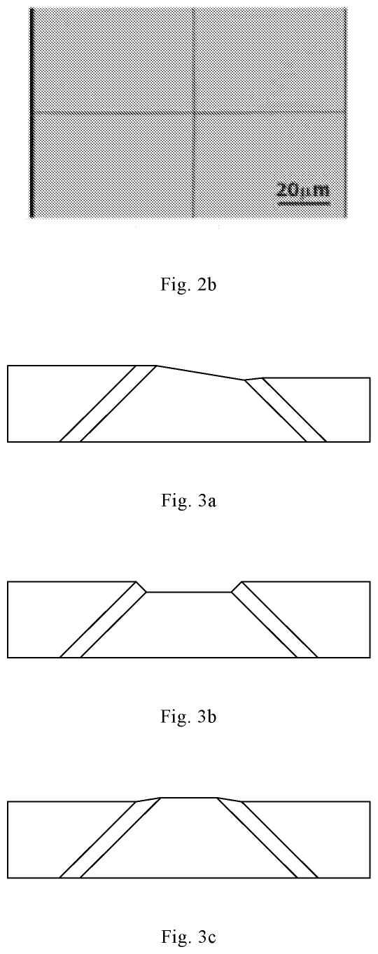

FIG. 2a illustrates a surface of a wafer near a scribing line after cut by a diamond slicer. As shown in FIG. 2a, after cut with the slicer, the cutting surface of the wafer has many burrs and is uneven. For the microfluidic device shown in FIG. 1, because the inlet and outlet of the fluid channel are located at the edge of the scribing line, slight defects may deteriorate the quality of the devices. In addition, in most scribing processes, the grooves formed by slicing may have a width substantially equal to that of the slicer, and thus many solid particles or debris may also be generated during the scribing process. When the inlet and outlet of the fluid channel are located at the edge of the scribing line, the inlet and outlet may be fluidly connected to the ambience after scribing, so the particles or debris generated in the scribing process may enter into the fluid channel through the open inlet and outlet, which is likely to block the fluid channel.

Another commonly used wafer scribing technology is laser scribing process. Compared with the mechanical scribing process, laser scribing can significantly reduce the scribing loss and debris after wafer scribing, as shown in FIG. 2b. However, the laser light source has limited energy and sometimes requires multiple times of cutting to complete device separation. In addition, for a composite wafer with a large thickness, it is required to apply scribing from the upper and lower surfaces of the composite wafer to an intermediate bonding surface within the wafer. Multiple cutting and secondary alignment inevitably introduce dis-alignment defects. Therefore, when the inlet and outlet of the microfluidic channel are located near the scribing line, the dis-alignment may directly change the lengths of the inlet and outlet of the microfluidic channel and the cross section of the microfluidic channel. In addition, the laser scribing process must apply an external force to split the devices after the scribing process, which may also cause slight damage at the interfaces between adjacent devices, and slight cracking burrs near the inlet and outlet of the channel may affect the integrity of the cross section of the nozzle. Therefore, the laser scribing process has limited improvement in the yield of microfluidic devices.

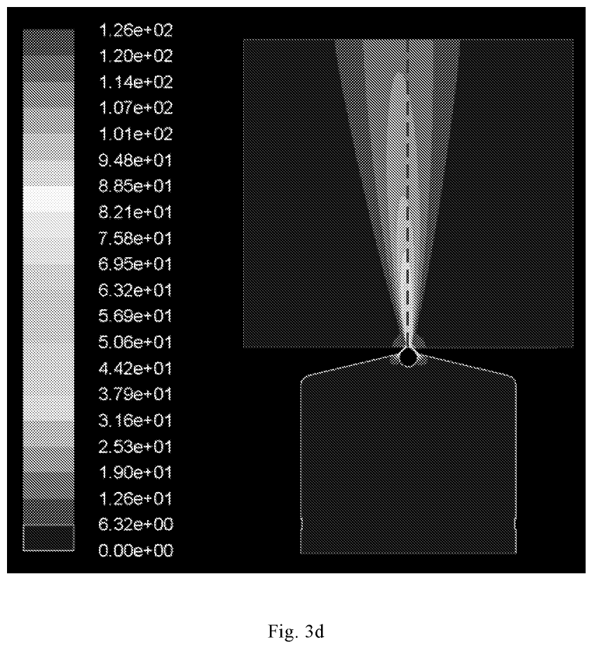

FIGS. 3a to 3c illustrate several schematic diagrams of uneven edges of fluid outlets caused by scribing defects. FIG. 3d illustrates a simulated profile of a spray emitted from a microfluidic device with the scribing defect shown in FIG. 3a.

In order to solve the device quality defects caused by the above scribing processes, after a lot of experiments and process verification, the inventor of the present application invented a new type of microfluidic device, which has an expansion groove(s) near an outlet and/or inlet of its fluid channel. The expansion groove can keep the cutting surface away from the outlet and/or inlet and avoid directly contacting with the outlet and/or inlet, so that the scribing process may not affect the profile of the outlet or inlet of the fluid channel. Therefore, the fluid channel of the microfluidic device obtained after scribing generally has an ideal shape that precisely matches design parameters, which can greatly reduce the quality defects of mass-produced devices.

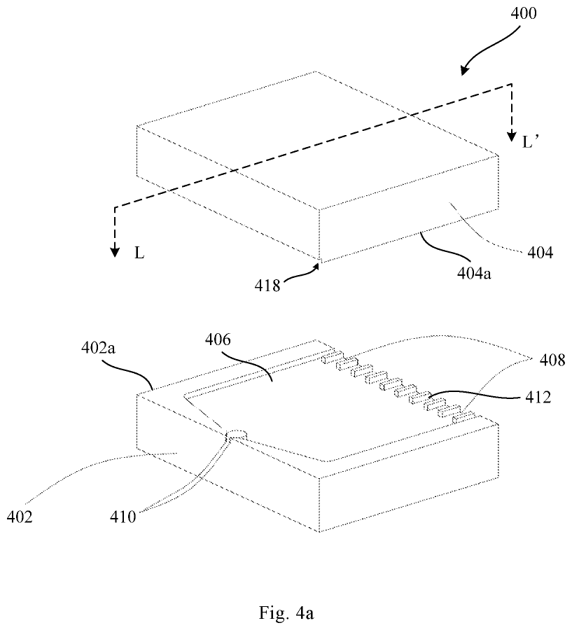

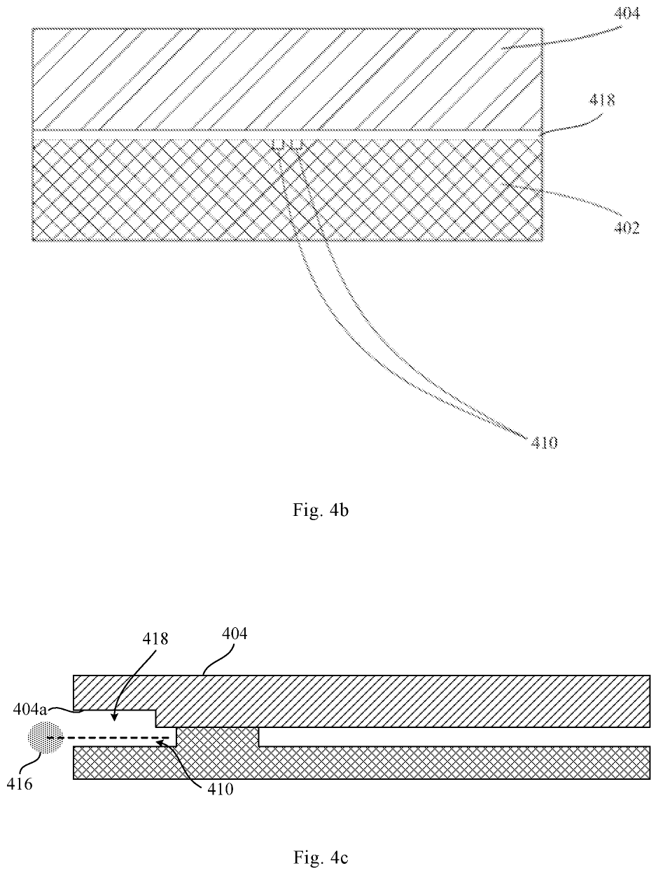

FIGS. 4a to 4c illustrate schematic diagrams of a microfluidic device 400 according to an embodiment of the present application. FIG. 4a is an exploded perspective view of the microfluidic device 400, and FIG. 4b is a cross-sectional view of the microfluidic device 400 at the fluid outlet.

As shown in FIG. 4a, the microfluidic device 400 includes a first substrate 402 and a second substrate 404. The first substrate 402 and the second substrate 404 have an assembling side 402a and an assembling side 404a, respectively, which can be connected to each other to assemble the first substrate 402 and the second substrate 404 together. In some embodiments, the substrates 402 and 404 may be silicon wafers, glass wafers, or wafers of other materials. For example, the first substrate 402 may be a silicon wafer, and the second substrate 404 may be a glass wafer. The two substrates 402 and 404 may be connected to each other by electrostatic bonding. For another example, both the first substrate 402 and the second substrate 404 may be silicon wafers, which may be connected to each other by silicon-silicon direct bonding or adhesive bonding.

The first substrate 402 has a fluid chamber channel 406 on its assembling side 402a. The fluid chamber channel 406 is recessed downward from the surface of the assembling side 402a by a certain depth. In some embodiments, a depth of the fluid chamber channel 406 is less than a thickness of the first substrate 402. In other embodiments, the depth of the fluid chamber channel may be equal to the thickness of the first substrate, that is, the fluid chamber channel penetrates through the first substrate; in this case, the microfluidic device may further include a third substrate which, together with the first substrate, enclose the fluid chamber channel from both sides of the first substrate, respectively. In some embodiments, the fluid chamber channel 406 may be formed by a plasma etching process or other similar processes.

Still referring to FIG. 4a, when the first substrate 402 and the second substrate 404 are connected to each other, the second substrate 404 generally closes the fluid chamber channel 406 from above the fluid chamber channel 406, thereby forming a fluid chamber having one or more fluid inlets 408 and a fluid outlet 410. When the microfluidic device 400 is in operation, liquid flows into the fluid chamber from the fluid inlets 408 and subsequently flows out of the fluid chamber via the fluid outlet 410. In the embodiment shown in FIG. 4a, the microfluidic device 400 is used as a liquid atomizer. Accordingly, the fluid chamber includes a plurality of fluid inlets 408, and the fluid inlets 408 are separated from each other by respective separation columns 412 therebetween. The separation column 412 allows the fluid flowing into the fluid chamber to form multiple flows, which is beneficial for reducing the size of the droplets before atomizing the liquid. In some embodiments, downstream of the separation columns 412, a one-stage or multi-stage filter structure (not shown in the figure) may also be provided in the fluid chamber. The filter structure can help to prevent solid particles in the liquid fluid from flowing into the fluid outlet 410 and blocking the fluid outlet 410, and can further help to further separate the liquid flows in the fluid chamber.

After flowing through the entire fluid chamber, the liquid may flow out of the chamber via the fluid outlet 410. In practical applications, depending on the pressure of the fluid, the fluid will be emitted from the fluid outlet 410 at a certain speed. FIG. 4c is a cross-sectional view of the microfluidic device 400 shown in FIG. 4a along line LL′ (through the fluid outlet). As shown in FIG. 4c, two jet flows are respectively sprayed out of the fluid chamber via the two fluid outlets 410, and meet at a convergence point 416. The two jet flows collide into each other at the convergence point 416, so that kinetic energy of the jet flows can break the flows. A diameter and cross-section of the fluid outlet 410 determine the flow rate of a single jet flow, and an angle between the two jet flows determines the fluid resistance for the fluid. The greater the angle is, the greater the fluid resistance is. In addition, an aspect ratio (a ratio of length to diameter) of the fluid channel connected to the fluid outlet 410 also affects the fluid resistance and flow rate. Therefore, in practical applications, parameters such as the length and diameter of the fluid channel, the diameter of the fluid outlet, and the spacing between the two fluid outlets need to be accurately designed to accurately determine the position of the convergence point of the two jet flows, and the size of liquid droplets and spray profile after the collision of the jet flows.

Still referring to FIG. 4c, the second substrate 404 has, on its assembling side 404a, an outlet expansion groove 418 adjacent to the fluid outlet 410. The outlet expansion groove 418 extends downstream from the fluid outlet 410, that is, generally extends in the direction of the liquid outflows. It can be seen that at the fluid outlet 410, the outer profile of the outlet expansion groove 418 is located outside the outer profile of the fluid outlet 410. For example, in the embodiment shown in FIG. 4a with two fluid outlets 410, both fluid outlets 410 are located within the outer profile of the outlet expansion groove 418, so that the wall of the outlet expansion groove 418 does not substantially affect the liquid flow emitted from the liquid outlet 410.

The fluid chamber in the microfluidic device 400 shown in FIG. 4a has two fluid outlets, and the jet flows passing through their respective flow paths can meet and collide with each other. In other embodiments, the fluid chamber may have one or more separate fluid outlets. In this case, each fluid outlet may have a corresponding outlet expansion groove. For example, both the fluid outlet 410 and the outlet expansion groove 418 may have a generally rectangular outer profile, and both of or at least one of the length and width of the outer profile of the outlet expansion groove 418 are greater than both of or the respective one of the length and width of the outer profile of the fluid outlet 410. For another example, the fluid outlet 410 and the outlet expansion groove 418 may each have a circular outer peripheral profile, and the diameter of the outer profile of the outlet expansion groove 418 may be larger than the diameter of the outer profile of the fluid outlet 410. Optionally, a plurality of mutually independent fluid outlets may also collectively be inside a single outlet expansion groove; in this case, at each fluid outlet, the outer profile of the outlet expansion groove is located outside the outer profiles of all the fluid outlets.

It should be further noted that, in the embodiment shown in FIG. 4c, the outlet expansion groove 418 has a substantially cubic shape, and its outer profile and cross-sectional shape at the fluid outlet 410 are the same as its outer profile and cross-sectional shape that is further downstream from the fluid outlet. In some other embodiments, the outer profile and cross-sectional shape of the outlet expansion groove 418 at the fluid outlet 410 may also be different from the outer profile and cross-sectional shape further downstream from the fluid outlet. For example, the outlet expansion groove 418 may have a flared structure that expands outward from the outlet 410, or any other similar and suitable structures.

It can be seen that the outlet expansion groove provided downstream of the fluid outlet can space the fluid outlet(s), which determines jet flow(s) (including shape, flow rate, speed and orientation), away from the edge of the microfluidic device, thereby effectively protecting the fluid outlet from being affected by scribing defects. In this way, the yield of mass-produced microfluidic devices can be significantly improved.

Still referring to FIG. 4c, the two jet flows of the fluid chamber are respectively sprayed out of the fluid chamber via the two fluid outlets 410 and converge at the convergence point 416. The convergence point 416 may be located outside the outlet expansion groove 418, for example, a few microns to hundreds of microns or even a few millimeters away from an end of the outlet expansion groove 418. This design can ensure that the spray formed by the convergence of the jet flows does not substantially (at least as little as possible) contact with the wall of the outlet expansion groove 418, thereby avoiding limitation or influence on the particle size of the atomized droplets in the spray by the outlet expansion groove 418.

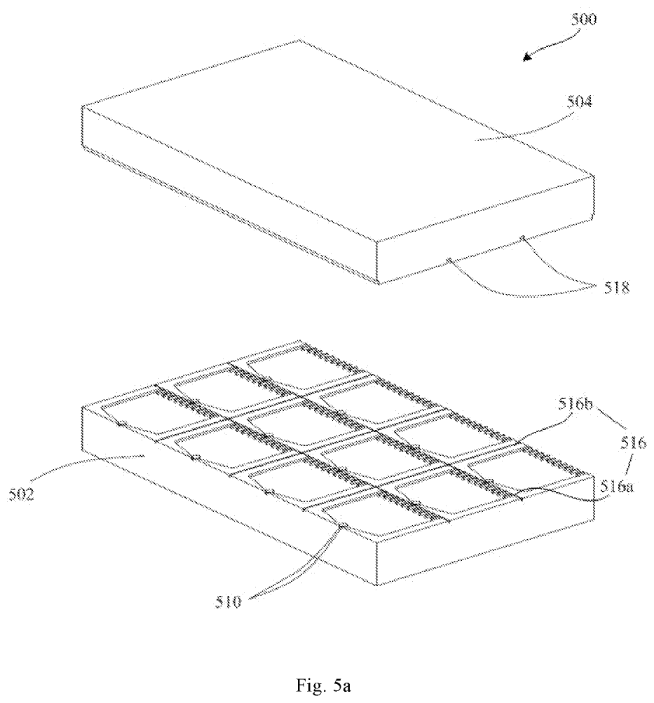

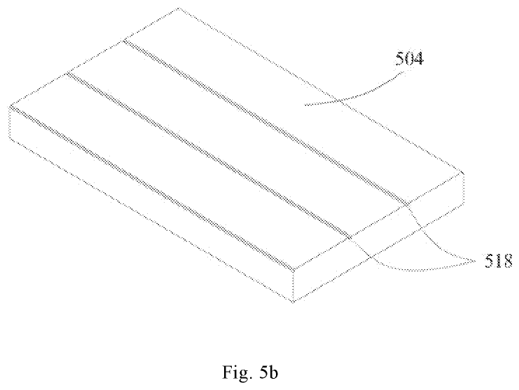

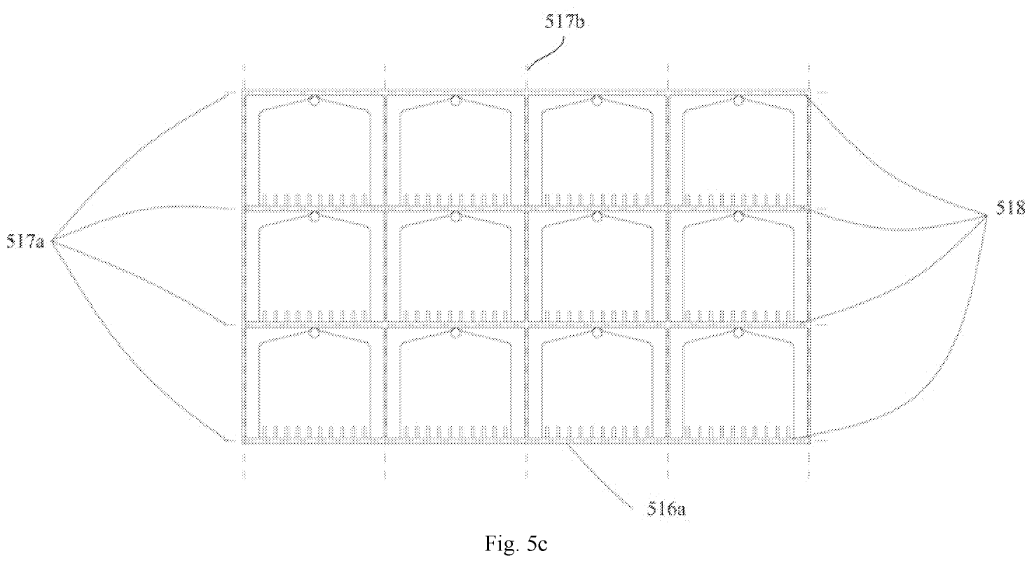

FIG. 5a illustrates perspective views of a first substrate and a second substrate when a wafer including a plurality of microfluidic devices as shown in FIG. 4a is not sliced. FIG. 5b illustrates that the first substrate and the second substrate shown in FIGS. 5a and 5b overlap with each other.

As shown in FIGS. 5a and 5c, a plurality of microfluidic devices are arranged in an array on the first substrate 502, and are separated from each other by a plurality of elongated scribing regions 516. The plurality of scribing regions 516 include first scribing regions 516a between the fluid inlets and outlets of the microfluidic devices, and second scribing regions 516b perpendicular to the first scribing regions 516a. Each scribing region has a central axis 517a or 517b. The second substrate 504 has a plurality of outlet expansion groove regions 518 formed on its assembling side 504a. These outlet expansion groove regions 518 are parallel to each other and are generally aligned with the first scribing regions 516a on the first substrate 502. In some embodiments, the outlet expansion groove region 518 may have a different length than the first scribing region 516a, but they are aligned with each other at least at the fluid outlet.

Still referring to FIG. 5c, when fabricating the microfluidic devices, after the outlet expansion groove regions 518 and the first scribing regions 516a are aligned with each other, the first substrate 502 and the second substrate 504 are connected to each other. In this way, the outlet expansion groove regions 518 are adjacent to and extend downstream from the respective fluid outlets 510. When scribing the connected first substrate 502 and second substrate 504, the outlet expansion groove regions 518 and the first scribing regions 516a are scribed to form respective outlet expansion grooves immediately downstream of the respective fluid outlets.

In some embodiments, each pair of the first scribing region 516a and the outlet expansion groove region 518 may have substantially the same width, so that the two regions substantially overlap with each other. For example, the width of the first scribing region 516a may be 30 um, that is, a distance between the fluid outlet of a microfluidic device and the fluid inlet of another microfluidic device adjacent thereto is 30 um. The width of the outlet expansion groove region 518 is also 30 um, so that distances between the central axis of an outlet expansion groove region 518 and a fluid inlet and a fluid outlet of adjacent fluidic devices are both 15 um. If a diamond slicer with a blade thickness of 10 um is used to scribe the substrate by aligning with the central axis of the scribing region, then the fluid inlet and the fluid outlet are both 10 um from the respective edges of the diamond slicer. Even assuming that there is a dis-alignment of 5 um, after cutting, the fluid inlet and fluid outlet defined by the outlet expansion groove region 518 are at least 5 um apart from the edge of scribing line. In other words, the end of the outlet expansion groove (located at the edge of the cutting line) is at least 5 um from the corresponding fluid outlet, which corresponds to the outer extension of the outlet expansion groove. It can be seen that since the outlet expansion groove has a certain external extension, the shape of the fluid outlet is essentially formed by the inner side of the outlet expansion groove on the first substrate (away from the edge of the cutting line) and the fluid chamber channel on the second substrate, rather than being defined by the edge of the scribing line and the fluid chamber channel. Therefore, the shape of the fluid outlet my not be affected by scribing stress or defects caused by particles, but can be consistent with the original parameters during device design.

FIG. 5c is a schematic diagram of the first scribing regions 516a and the outlet expansion groove regions 518 separated by a single time of scribing. In other embodiments, the first scribing regions and the outlet expansion groove regions may be separated by multiple times of scribing. For example, the first scribing regions 516a and the outlet expansion groove regions 518 may each have a width of, for example, 200 um. Assuming that the design value of the outer extension of the outlet expansion groove is 10 um, a diamond slicer may cut the first scribing region 516a and the outlet expansion groove region 518 at a location of 15 um away from the fluid outlet and 15 um away from the fluid inlet. It can be seen that, in the case where the first scribing region is too wide and multiple times of scribing is required to cut the wafer, the extension of the outlet expansion groove extending from the fluid outlet mainly depends on the location of the scribing operation closest to the fluid outlet.

Based on a similar concept, in addition to the outlet expansion groove at the fluid outlet, an inlet expansion groove may also be disposed at the fluid inlet, and the inlet expansion groove may also keep the fluid inlet relatively away from the scribing line.

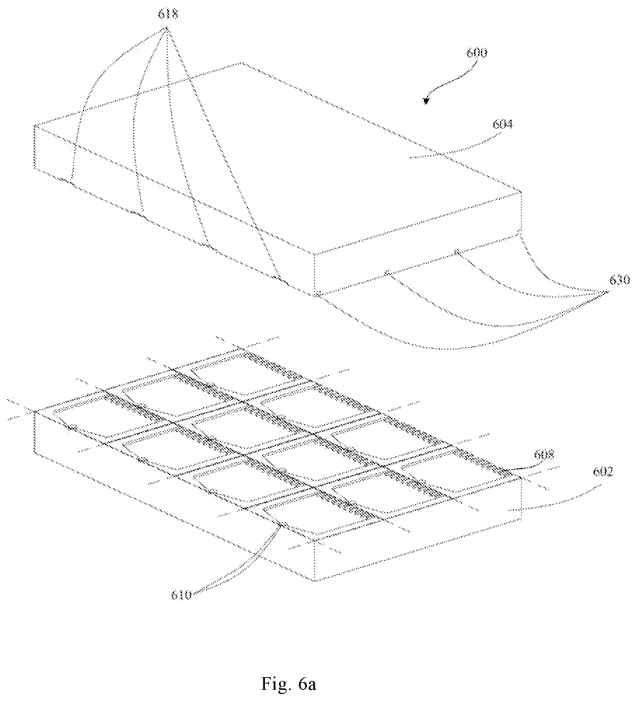

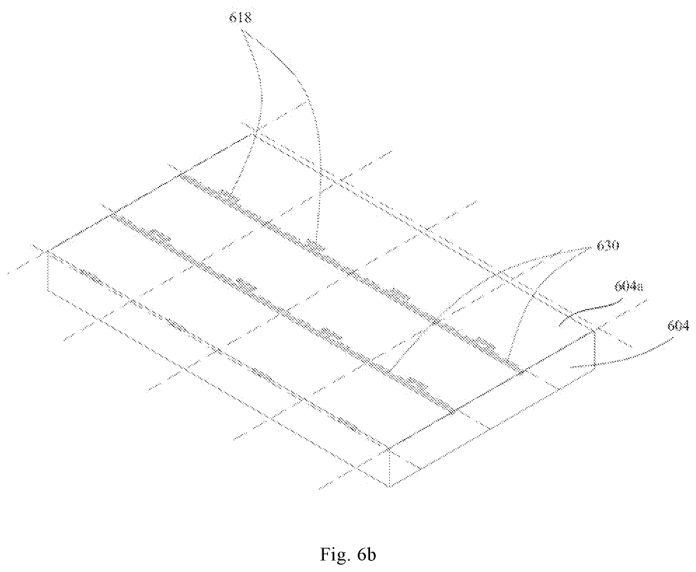

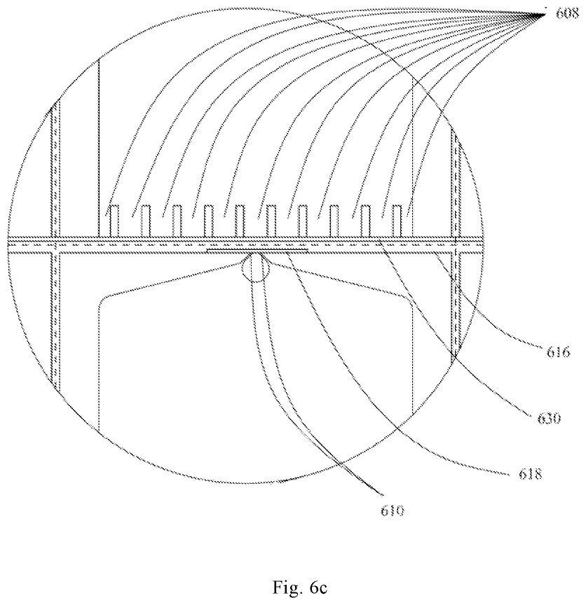

FIGS. 6a to 6c illustrate schematic diagrams of a microfluidic device 600 according to another embodiment of the present application.

As shown in FIGS. 6a to 6c, unlike the embodiment shown in FIG. 4a, the microfluidic device 600 has an outlet expansion groove 618 and an inlet expansion groove 630 on an assembling side 604a of a second substrate 604. When not scribed, both the outlet expansion groove 618 and the inlet expansion groove 630 are located in a scribing region 616. After the second substrate 604 and the first substrate 602 having the fluid chamber groove 606 are aligned and connected with each other, the outlet expansion groove 618 is adjacent to the fluid outlet 610, the inlet expansion groove 630 is adjacent to the fluid inlet 608, and the inlet expansion groove 630 extends upstream from the fluid inlet 608. At the fluid inlet 608, the outer profile of the inlet expansion groove 630 is outside the outer profile of the fluid inlet 608. Similar to the outlet expansion groove 618, the inlet expansion groove 630 can keep the fluid inlet 608 away from the scribing line to avoid cutting stress or particle-induced defects from affecting the shape of the fluid inlet.

As shown in FIG. 6b, on the assembling side 604a of the second substrate 604, the inlet expansion groove 630 generally spans across the second substrate 604, and the outlet expansion groove 618 has a relatively narrower width because the overall width of the fluid inlet is large and the width of the fluid outlet is narrow. It can be understood that in practical applications, the outer profile of the outlet expansion groove 618 may be outside the outer profile of the fluid outlet at the fluid outlet, and the specific length and width can be designed and adjusted as desired.

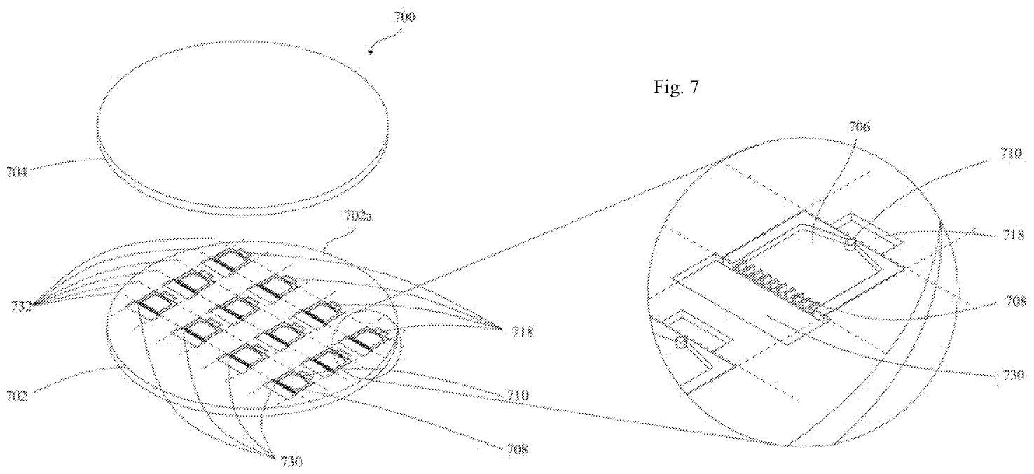

FIG. 7 illustrates a schematic diagram of a microfluidic device 700 according to another embodiment of the present application.

As shown in FIG. 7, a fluid chamber channel 706 of the microfluidic device 700 is formed on an assembling side 702a of a first substrate 702. In addition, an inlet expansion groove 730 adjacent to a fluid inlet 708 and an outlet expansion groove 718 adjacent to a fluid outlet 710 are also disposed on the assembling side 702a. When viewed from the assembling side 702a, both the inlet expansion groove 730 and the outlet expansion groove 718 have a pocket structure. The inlet expansion groove 730 has a width greater than that of the fluid inlet 708, and extends upstream from the fluid inlet 708. The outlet expansion groove 718 has a width greater than that of the fluid outlet 710, and extends downstream from the fluid outlet 710.

In some embodiments, the depth of the inlet expansion groove 730 and the outlet expansion groove 718 can be greater than the depth of the fluid chamber channel 706 to prevent their walls from blocking the liquid flow into or out of the fluid chamber channel 706. In actual processing, the fluid chamber channel as well as the inlet expansion groove and/or outlet expansion groove can be selectively etched with different depth by, for example, a plasma etching process.

Similarly, the extension length of the inlet expansion groove 730 and the outlet expansion groove 718 depend on the location of the scribing line 732, and are not repeated here.

Although the embodiments shown in FIGS. 4a-4c and FIG. 7 respectively form outlet expansion groove(s) and/or inlet expansion groove(s) on one of the assembling side of the first substrate and the assembling side of the second substrate, in practical applications, it is possible that the outlet expansion groove and/or the inlet expansion groove are both formed on the assembling sides of the two substrates as desired. The outlet expansion grooves on both assembling sides may both be adjacent to the fluid outlet and aligned with each other at least at the fluid outlet. Similarly, the inlet expansion grooves on both assembling sides may be adjacent to the fluid inlet and aligned with each other at least at the fluid inlet.

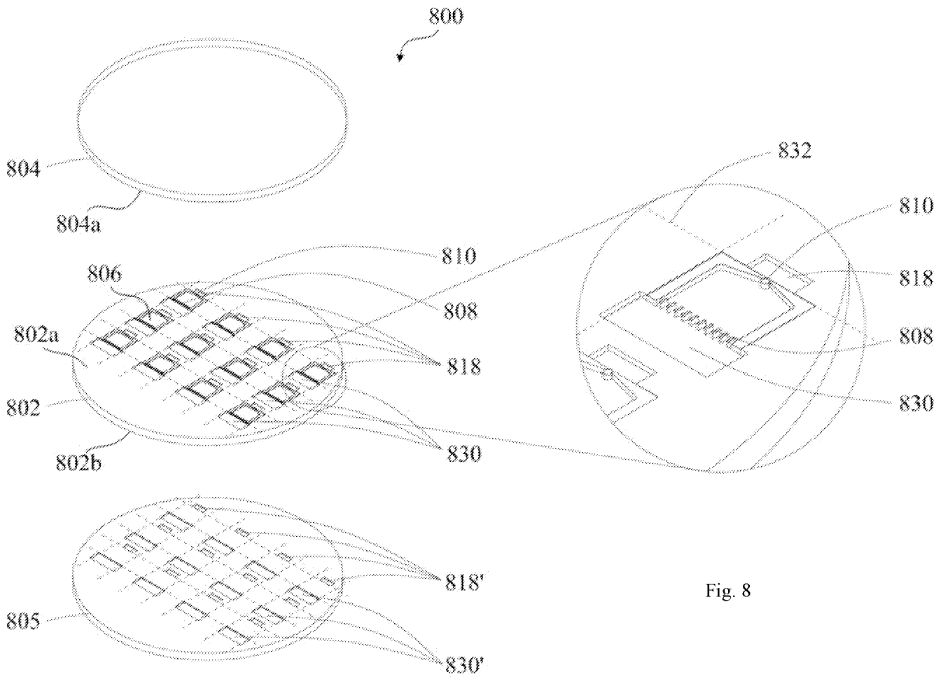

FIG. 8 illustrates a schematic diagram of a microfluidic device 800 according to another embodiment of the present application.

As shown in FIG. 8, the microfluidic device 800 is formed of a first substrate 802, a second substrate 804 and a third substrate 805. The first substrate 802 is formed with a fluid chamber channel 806 on both sides thereof (only the fluid chamber channel on a first side 802a are shown in the figure). In addition, inlet expansion grooves 830 and outlet expansion grooves 818 are also formed on the first side 802a, while the inlet expansion grooves and the outlet expansion grooves are not formed on the assembling side 804a of the second substrate 804. In contrast, the inlet expansion grooves and the outlet expansion grooves are not formed on the second side 802b, but the inlet expansion grooves 830′ and the outlet expansion grooves 818′ are formed on the assembling side 805a of the third substrate 805. In this way, after the three substrates are connected together, the fluid chamber channels on the first side 802a and the second side 802b both have upstream and downstream expansion grooves, thereby the fluid inlet and the fluid outlet can be kept away from directly adjacent to the scribing lines. The extension lengths of the outlet expansion grooves and the inlet expansion grooves can vary depending on the location of the scribing line 832.

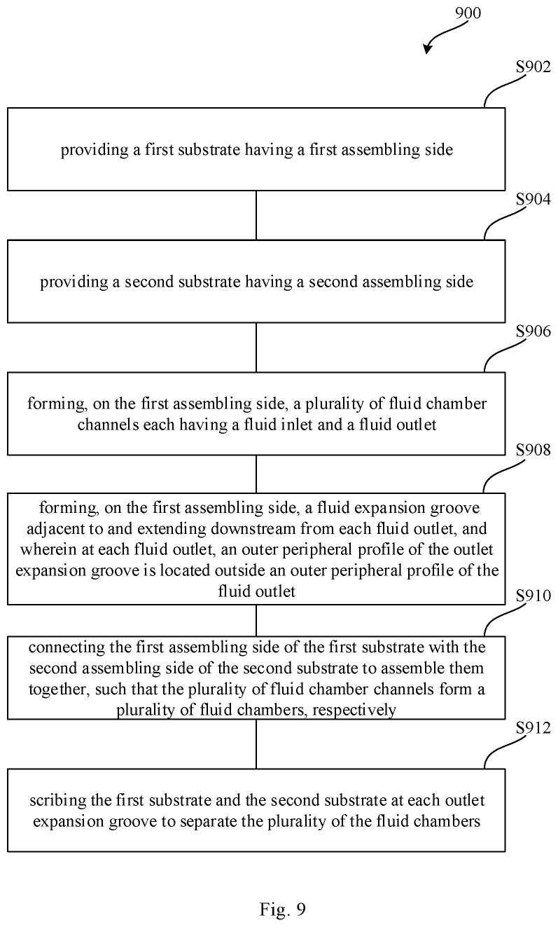

FIG. 9 illustrates a method of manufacturing a microfluidic device according to an embodiment of the present application.

As shown in FIG. 9, the manufacturing method includes: in step S902, providing a first substrate having a first assembling side; in step S904, providing a second substrate having a second assembling side; in step S906, forming, on the first assembling side, a plurality of fluid chamber channels each having a fluid inlet and a fluid outlet; in step S908, forming, on the first assembling side, a fluid expansion groove adjacent to and extending downstream from each fluid outlet, and wherein at each fluid outlet, an outer peripheral profile of the outlet expansion groove is located outside an outer peripheral profile of the fluid outlet; in step S910, connecting the first assembling side of the first substrate with the second assembling side of the second substrate to assemble them together, such that the plurality of fluid chamber channels form a plurality of fluid chambers, respectively; and in step S912, scribing the first substrate and the second substrate at each outlet expansion groove to separate the plurality of the fluid chambers.

In some embodiments, each of the plurality of fluid chambers has a plurality of fluid outlets, and at each fluid outlet of the plurality of fluid outlets, the outer peripheral profile of the outlet expansion groove is located outside the outer peripheral profile of the fluid outlet.

In some embodiments, the plurality of fluid outlets have respective fluid spraying directions that converge together.

In some embodiments, the respective fluid spraying directions of the plurality of fluid outlets have a convergence point located outside of the outlet expansion groove.

In some embodiments, a depth of the outlet expansion groove is greater than a depth of the fluid chamber channel on the same substrate.

In some embodiments, a width of the outlet expansion groove is greater than a width of the fluid chamber channel on the same substrate.

In some embodiments, the method further comprises: forming, on the second assembling side, another outlet expansion groove aligned with the outlet expansion groove of the first assembling side at least at the fluid outlet.

In some embodiments, the method further comprises: forming, on the first assembling side, an inlet expansion groove adjacent to and extending upstream from the fluid inlet, and wherein at the fluid inlet, an outer peripheral profile of the inlet expansion groove is located outside an outer peripheral profile of the fluid inlet.

In some embodiments, the fluid chamber has a plurality of fluid inlets, and at each fluid inlet of the plurality of fluid inlets, the outer peripheral profile of the inlet expansion groove is located outside the outer peripheral profile of the fluid inlet.

For specific details of the manufacturing method of the microfluidic device of the present application, reference may be made to the details of the microfluidic device of the present application, which will not be repeated here.

The microfluidic device of the present application can be used in various scenarios that require precise fluid control, especially used as a liquid atomizer.

It should be noted that although several modules or sub-modules of the microfluidic device are mentioned in the above detailed description, this division is merely exemplary and not mandatory. In fact, according to the embodiments of the present application, the features and functions of the two or more modules described above may be embodied in one module. Conversely, the features and functions of a module described above can be further divided into multiple modules to be embodied.

Those of ordinary skill in the art can understand and implement other changes to the disclosed embodiments by studying the description, the disclosure, the drawings, and the appended claims. In the claims, the word “comprising” does not exclude other elements and steps, and the words “a” and “an” do not exclude plurals. In the actual application of this application, one part may perform the functions of multiple technical features cited in the claims. Any reference signs in the claims should not be construed as limiting the scope.

1. A microfluidic device comprising:
a first substrate having a first assembling side; and
a second substrate having a second assembling side connectable with the first assembling side to assemble the first substrate and the second substrate together;
wherein at least one of the first assembling side and the second assembling side has a fluid chamber channel, and after the first substrate and the second substrate are connected together, the fluid chamber channel forms a fluid chamber having a fluid inlet and a fluid outlet; and
wherein the at least one of the first assembling side and the second assembling side having the fluid chamber channel has an outlet expansion groove adjacent to and extending downstream from the fluid outlet, and wherein at the fluid outlet, an outer peripheral profile of the outlet expansion groove is located outside an outer peripheral profile of the fluid outlet.

2. The microfluidic device of claim 1, wherein the fluid chamber has a plurality of fluid outlets, and at each fluid outlet of the plurality of fluid outlets, the outer peripheral profile of the outlet expansion groove is located outside the outer peripheral profile of the fluid outlet.

3. The microfluidic device of claim 2, wherein the plurality of fluid outlets have respective fluid spraying directions that converge together.

4. The microfluidic device of claim 3, wherein the respective fluid spraying directions of the plurality of fluid outlets has a convergence point located outside of the outlet expansion groove.

5. The microfluidic device of claim 1, wherein a depth of the outlet expansion groove is greater than a depth of the fluid chamber channel on the same substrate.

6. The microfluidic device of claim 1, wherein a width of the outlet expansion groove is greater than a width of the fluid chamber channel on the same substrate.

7. The microfluidic device of claim 1, wherein the fluid chamber has a filter therein.

8. The microfluidic device of claim 1, wherein the first assembling side and the second assembling side have outlet expansion grooves aligned with each other at least at the fluid outlet.

9. The microfluidic device of claim 1, wherein the at least one of the first assembling side and the second assembling side having the fluid chamber channel has an inlet expansion groove adjacent to and extending upstream from the fluid inlet, and wherein at the fluid inlet, an outer peripheral profile of the inlet expansion groove is located outside an outer peripheral profile of the fluid inlet.

10. The microfluidic device of claim 9, wherein the fluid chamber has a plurality of fluid inlets, and at each fluid inlet of the plurality of fluid inlets, the outer peripheral profile of the inlet expansion groove is located outside the outer peripheral profile of the fluid inlet.

11. A fluid atomizer comprising the microfluidic device according to claim 1.

12. A method for manufacturing a microfluidic device, the method comprising:
providing a first substrate having a first assembling side;
providing a second substrate having a second assembling side;
forming, on the first assembling side, a plurality of fluid chamber channels each having a fluid inlet and a fluid outlet;
forming, on the first assembling side, a fluid expansion groove adjacent to and extending downstream from each fluid outlet, and wherein at each fluid outlet, an outer peripheral profile of the outlet expansion groove is located outside an outer peripheral profile of the fluid outlet;
connecting the first assembling side of the first substrate with the second assembling side of the second substrate to assemble them together, such that the plurality of fluid chamber channels form a plurality of fluid chambers, respectively; and
scribing the first substrate and the second substrate at each outlet expansion groove to separate the plurality of the fluid chambers.

13. The method of claim 12, wherein each of the plurality of fluid chambers has a plurality of fluid outlets, and at each fluid outlet of the plurality of fluid outlets, the outer peripheral profile of the outlet expansion groove is located outside the outer peripheral profile of the fluid outlet.

14. The method of claim 13, wherein the plurality of fluid outlets have respective fluid spraying directions that converge together.

15. The method of claim 14, wherein the respective fluid spraying directions of the plurality of fluid outlets have a convergence point located outside of the outlet expansion groove.

16. The method of claim 12, wherein a depth of the outlet expansion groove is greater than a depth of the fluid chamber channel on the same substrate.

17. The method of claim 12, wherein a width of the outlet expansion groove is greater than a width of the fluid chamber channel on the same substrate.

18. The method of claim 12, further comprising:
forming, on the second assembling side, another outlet expansion groove aligned with the outlet expansion groove of the first assembling side at least at the fluid outlet.

19. The method of claim 12, further comprising:
forming, on the first assembling side, an inlet expansion groove adjacent to and extending upstream from the fluid inlet, and wherein at the fluid inlet, an outer peripheral profile of the inlet expansion groove is located outside an outer peripheral profile of the fluid inlet.

20. The method of claim 19, wherein the fluid chamber has a plurality of fluid inlets, and at each fluid inlet of the plurality of fluid inlets, the outer peripheral profile of the inlet expansion groove is located outside the outer peripheral profile of the fluid inlet.
